(19) United States
(12) Patent Application Publication
(10) Pub. No.: US 2021/0222207 A1
(43) Pub. Date: Jul. 22, 2021

(54) METHOD FOR PRODUCING ETHANOL FUEL BY USING ELAEAGNUS ANGUSTIFOLIA L. AS RAW MATERIAL

(71) Applicant: Heilongjiang Splendid Land Biotech Co., Ltd, Daqing, Heilongjiang (CN)

(72) Inventors: Liqiang LIU, Daqing, Heilongjiang (CN); Xiaoyan LV, Daqing, Heilongjiang (CN); Lin LIU, Daqing, Heilongjiang (CN)

(21) Appl. No.: 17/059,106

(22) PCT Filed: May 22, 2019
(86) PCT No.: PCT/CN2019/087919

§ 371 Date: Nov. 25, 2020

(30) Foreign Application Priority Data
May 25, 2018 (CN) 201810520574.X

(51) Int. Cl. C12P 7/06; C07C 29/80; C12P 7/14

(52) U.S. Cl. CPC C12P 7/06; C12P 7/14; C07C 29/80

(57) ABSTRACT

A process method is used for producing ethanol fuel by using Elaeagnus angustifolia L. as a raw material. The method includes pretreatment and pulping of Elaeagnus angustifolia L., solid-liquid separation or liquefaction treatment, fermentation, and distillation and dehydration of ethanol. The present invention produces ethanol by fermentation with Elaeagnus angustifolia L. as a raw material, the process method is simple, and the sugar-alcohol conversion efficiency is basically the same as when fermented with corn and other starches as raw materials, so that a new non-grain biological resource-Elaeagnus angustifolia L. is used to produce liquid bioenergy without consuming food resources, which broadens the types of raw materials for production of ethanol fuel.

### FIELD OF THE APPLICATION

The present invention relates to the field of ethanol production, in particular, to a method for producing ethanol fuel by using Elaeagnus angustifolia L. as a raw material.

### BACKGROUND TECHNOLOGY

As a food and chemical raw material, ethanol has always been the leading product in the fermentation industry. For a long time, ethanol has been produced by microbial fermentation, and the raw materials are mostly crops such as corn, wheat, sweet sorghum, cassava, and sweet potatoes. For example, Appl. No. 201410662601.9 disclosed a method for preparing ethanol, including: crushing starch raw materials to form large granular starch raw materials; adding water and high temperature resistant amylase to the large-granular starch material and mixing and stirring to form a first mash; gelatinizing the first mash to form a second mash; liquefaction treatment; saccharifying the second mash that has been subjected to the liquefaction treatment to form a third mash; fermenting the third mash. According to the method of the present invention, the viscosity of the starch raw material mash can be controlled within the acceptable range of the process in the case of using starch raw material with larger granularity. And, since the viscosity and liquid saccharification effect of large-granular starch mash are well controlled, the problems of transportation difficulties and incomplete liquid saccharification that occur when large-granular starch mash is used for ethanol production in the prior art are effectively solved, so as to further reduce energy consumption in the production process.

Moreover, corn stalks were also used as raw materials to produce ethanol; for example, Tianjin University registered Appl. No. 201711404719.1 in 2017 (the name of the invention is the process of producing ethanol from corn stalks), but the conversion rate is relatively low, and there are equipment corrosion problems, and the possibility of industrialization is relatively weak at present. At present, the basic national conditions of my country with a large population and a small amount of arable land severely restrict the large-scale development of the industrial production of ethanol fuel from starch raw materials such as corn, wheat and cassava, and the production of ethanol fuel, which uses corn and other grains as raw materials, is competing with people and animals for food, leading to unsustainable development. The “National Renewable Energy Medium and Long-term Development Plan” puts forward the development goal of biofuel ethanol and ethanol gasoline for vehicles to reach 10 million tons per year by 2020. In 2017, 15 departments including the National Development and Reform Commission and the National Energy Administration jointly issued the “Implementation Plan on Expanding Biofuel Ethanol Production and Promoting the Use of Ethanol-Added Gasoline for Vehicles”, which clearly expands the production of ethanol biofuel and promotes the use of ethanol-added gasoline for vehicles, should be based on domestic supply on the basis of ensuring national food security, promotes the production of bio-fuel ethanol and the promotion and use of ethanol gasoline for vehicles in a scientific and orderly manner, focuses on dealing with overdue and over-standard grains, strengthens the ability to control the grain market and enhances the level of quality and safety; the plan focuses on improving the comprehensive utilization of agricultural and forestry waste resources and promoting the development of advanced bioenergy industry; the plan focuses on implementing the strategic deployment of Northeast Revitalization, building a bio-fuel ethanol industrial base, and making new contributions to promoting my country's substitution of fossil energy, reducing greenhouse gas emissions, and cultivating new drivers of agricultural and rural development. By 2020, the use of ethanol-added gasoline is promoted for vehicles nationwide and basically achieves full coverage, and a market-oriented operation mechanism is initially established, and an advanced bio-liquid fuel innovation system is initially established.

Therefore, exploring new sources of non-grain biomass raw materials to replace corn and other food crops to produce ethanol fuel without competition with human beings for food and competition with food for the land while not destructing ecological environment is the basic policy for the development of bioethanol industry in our country and the direction for the development of the rural economy.

Elaeagnus angustifolia L. has aliases of Elaeagnus angustifolia L. (“Atlas of woody plants in Northeast China”), Elaeagnus angustifolia Linn. (Henan), Salix leucopi thecia (Liaoning), etc. This tree species grows in semi-arid, arid, semi-desert, and desert areas, and is distributed in the northwest desert, semi-desert area, North China, Shandong and Northeast China. Elaeagnus angustifolia L. is one of the few tree species that can survive on the Gobi Desert, and is known as the “treasure tree” in the desert and saline-alkali land.

Everything about Elaeagnus angustifolia L. is useful, wherein the leaves contain 4% protein, 2.4% crude fat, and 15.7% sugar. It is a high-quality fodder. For Elaeagnus angustifolia L., the pulp powder contains 6.76-7.94% crude protein, 1.34% crude fat, and 43-59% sugar. It can be used for non-staple food processing and can replace grain. Elaeagnus angustifolia L.'s flowers are fragrant, which can be extracted for aromatic oils and is a good source of honey. Elaeagnus angustifolia L.'s root has rhizobia, which can fix nitrogen and improve the soil. The seed kernel of has Elaeagnus angustifolia L. an oil content of 20.69%, which can be used to produce clean fuel (biodiesel); Elaeagnus angustifolia L. can be used to create economic forests and protection forests of Elaeagnus sylvestris, and can also cultivate scenic forests and green and beautify cities. In view of the fact that Elaeagnus angustifolia L. as a plant has a good prospect in terms of restoration of vegetation and afforestation and the sugar, protein and vitamins contained in the fruit of Elaeagnus angustifolia L. can meet the needs of yeast growth and fermentation, thus Elaeagnus angustifolia L. is a good raw material for the fermentation and production of ethanol fuel theoretically.

In order to solve the problem of the limited development of the existing industrial scale development of producing fuel ethanol from starch raw materials such as corn, wheat and cassava, the applicant researches and develops a process method for producing ethanol fuel by using Elaeagnus angustifolia L. as a raw material.

### SUMMARY OF INVENTION

The present invention provides a method for producing ethanol fuel by using Elaeagnus angustifolia L. as a raw material.

The technical solution of the present invention includes steps of:
pretreatment and pulping of Elaeagnus angustifolia L., liquefaction treatment, fermentation, and distillation and dehydration of ethanol; enucleating Elaeagnus angustifolia L., adding process water with a pH of 3.5 to 5.0 for pulping to obtain a jujube pulp; performing liquefaction or solid-liquid separation for the jujube pulp; adding a clear mash into a fermentation tank, adding antibiotic, and inoculating yeast wine or active dry yeast; fermenting to obtain a fermented and matured mash; distilling and dehydrating the matured mash, then condensing to obtain an ethanol liquid, followed by adding a deanaturing agent, to obtain a finished ethanol fuel.
Preferably, a method for producing ethanol fuel by using Elaeagnus angustifolia L. as a raw material includes steps of:

(1) pretreatment, including removing impurities and kernel of Elaeagnus angustifolia L., adding process water with a pH of 3.5 to 5.0 for pulping to obtain a jujube pulp, and controlling a mass concentration and a reducing sugar degree of the jujube pulp to 15% to 30% and to 15% to 20%;

(2) liquefaction treatment, including performing solid-liquid separation for the pulp, and adding the clear mash into the fermentation tank, wherein the solids are composted or dried to produce a fodder;

preferably, the liquefaction treatment includes cooking the jujube pulp, and the liquefaction treatment refers to the process of dissolving the sugar contained in the Elaeagnus angustifolia L. from solid sugar state into liquid sugar;

wherein the cooking includes: heating the jujube pulp to 85° C. to 105° C. through a multi-stage ejector and sending to a cooking kettle for cooking and keeping warm for 30 min to 180 min; cooling the jujube pulp mash to 20° C. to 35° C. through a heat exchanger;

preferably, the liquefaction treatment includes enzymatic hydrolysis and cooking treatment by adding pectinase and other viscosity reducing enzymes to the jujube pulp, with a process of preheating the jujube pulp to 30° C. to 60° C. through a first-stage ejector, adding pectinase in a proportion of 0.05% to 0.1% and keeping warm for 1 hour to 2 hours, then heating the jujube pulp to 85° C. to 105° C. through a multi-stage ejector and sending to a cooking kettle for cooking and keeping warm for 30 min to 180 min, to obtain the jujube pulp mash; cooling the jujube pulp mash to 20° C. to 35° C. through a heat exchanger;

the preheating the jujube pulp may include primary steam preheating directly, or also preheating by heat exchange of the mash using waste heat recovered from the cooling process, or preheating with waste heat recovered from the distillation and dehydration section;

(3) fermentation, including: adding the jujube pulp clear mash directly into the fermentation tank, adding yeast to ferment to produce alcohol in the fermentation tank, and passing sterile air through the fermentation process to maintain the vitality of the yeast, to obtain a matured mash;

wherein a time for the fermentation of the mash in the fermentation tank is 36 hours to 60 hours; an alcohol content of the fermented and matured mash reaches 7-11% (v/v); during the fermentation process, heat will be released, and a fermentation temperature may be stabilized by cooling outside the tank; the fermentation temperature is 28° C. to 35° C.; the fermentation tank employs a vertical agitator;

further, the time for the fermentation of the mash in the fermentation tank is 36 hours to 48 hours;

(4) distillation and dehydration of ethanol: preheating the matured mash of the section (3) to above 70° C. and sending to the distillation workshop, and then extracting an alcohol with a volume fraction of 95% from a liquid phase of a rectification tower as an anhydrous raw material after differential distillation;

preheating the anhydrous raw material and sending to a molecular sieve system for dehydration after passing through an evaporator and a superheater to obtain an ethanol fuel gas, and then cooling the ethanol fuel gas to obtain an ethanol fuel with a mass fraction of 99.5%;

adding a deanaturing agent into the ethanol fuel with a mass fraction of 99.5% to obtain the finished ethanol fuel, wherein the product quality meets the national standard GB18350-2013;

preferably, the whole process of the present invention adopts waste heat recovery technology to recover waste heat in distillation and dehydration sections, and is used for preheating anhydrous raw materials or heating mash or matured mash.

The present invention further provides a process method for producing ethanol fuel by using Elaeagnus angustifolia L. as a raw material, which includes steps of pretreatment and pulping of Elaeagnus angustifolia L., liquefaction treatment, fermentation, distillation and dehydration of ethanol.

Preferably, a method for producing ethanol fuel by using Elaeagnus angustifolia L. as a raw material includes steps of:

(1) pretreatment, including removing impurities and kernel of Elaeagnus angustifolia L., adding process water with a pH of 3.5 to 5.0 for pulping to obtain a jujube pulp, and controlling a mass concentration and a reducing sugar degree of the jujube pulp to 15% to 30% and to 15% to 20%, wherein the jujube pulp within the range may be directly applied to the fermentation of raw materials without adding amylase and saccharification enzyme for liquid saccharification in the subsequent fermentation stages, thereby simplifying steps and reducing costs;

(2) liquefaction treatment, including performing solid-liquid separation for the pulp, and adding the clear liquid into the fermentation tank for fermentation, wherein the solids are composted or dried to produce a fodder;

the solid-liquid separation refers to the process of separating insoluble matter such as bark and fiber from the jujube pulp;

the solid-liquid separation includes a single beating, grinding, washing, and separation processing;

further, the solid-liquid separation includes multiple processing steps such as beating, grinding, washing, and separation by one or more times;

preferably, the solid-liquid separation refers to a process with steps such as multiple grinding, washing, and separating using a filter press and/or a butterfly centrifuge and/or a double cone centrifuge and/or a decanter centrifuge.

preferably, the liquefaction treatment includes enzymatic hydrolysis and cooking treatment by adding pectinase and other viscosity reducing enzymes to the jujube pulp, with a process of preheating the jujube pulp to 30° C. to 60° C. through a first-stage ejector, adding pectinase in a proportion of 0.05% to 0.1% and keeping warm for 1 hour to 2 hours, then heating the jujube pulp to 85° C. to 105° C. through a multi-stage ejector and sending to a cooking kettle for cooking and keeping warm for 30 min to 180 min, to obtain the jujube pulp mash; cooling the jujube pulp mash to 20° C. to 35° C. through a heat exchanger;

preferably, the liquefaction treatment further includes enzymatic hydrolysis, cooking, solid-liquid separation, multi-effect evaporation and concentration by adding pectinase and other viscosity reducing enzymes to the jujube pulp, with a process of preheating the jujube pulp to 30° C. to 60° C. through a first-stage ejector, adding pectinase in a proportion of 0.05% to 0.1% and keeping warm for 1 hour to 2 hours, then heating the jujube pulp to 85° C. to 105° C. through a multi-stage ejector and sending to a cooking kettle for cooking and keeping warm for 30 min to 180 min; performing multiple grinding, washing, and separating on the jujube pulp, collecting the clear liquid, concentrating by multi-effect evaporation, and sending the concentrated mash directly to the fermentation tank, wherein after the separation, the solids are composted or dried to produce a fodder;

the preheating the jujube pulp may include primary steam preheating directly, or also preheating by heat exchange of the mash using waste heat recovered from the cooling process, or preheating with waste heat recovered from the distillation and dehydration section;

the multi-effect evaporative concentration refers to the use of a multi-effect evaporator such as an one-effect or more-effect evaporator to evaporate and concentrate the clear liquid;

(3) fermentation, including: adding the jujube pulp clear mash directly into the fermentation tank, adding yeast to ferment to generate the matured mash in the fermentation tank, and passing sterile air through the fermentation process to maintain the vitality of the yeast;

(4) distillation and dehydration of ethanol: preheating the matured mash that has been subjected to the fermentation to above 70° C. and sending to the distillation workshop, and then extracting an alcohol with a volume fraction of 95% from a liquid phase of a rectification tower as an anhydrous raw material after differential distillation;

preheating the anhydrous raw material and sending to a molecular sieve system for dehydration after passing through an evaporator and a superheater to obtain an ethanol fuel gas, and then cooling the ethanol fuel gas to obtain an ethanol fuel with a mass fraction of 99.5%;

adding a deanaturing agent into the ethanol fuel with a mass fraction of 99.5% to obtain the finished ethanol fuel, wherein the product quality meets the national standard GB18350-2013;

preferably, the whole process of the present invention adopts waste heat recovery technology to recover waste heat in distillation and dehydration sections, and is used for preheating anhydrous raw materials or heating mash or matured mash.

Compared to the prior art, the present invention has the following advantages and effects:
1, the sugar content of Elaeagnus angustifolia L. is very high, which is easy to be used by yeast. Elaeagnus angustifolia L. is rich in protein, vitamins and mineral elements, which is conducive to the fermentation and growth of yeast.
2, Due to the high content of fermentable sugar in Elaeagnus angustifolia L., the process does not need to add amylase and saccharification enzyme for liquid saccharification, and raw material fermentation technology may also be directly applied, which saves energy and greatly simplifies the production process.
3, In the process of the present invention, differential pressure distillation technology, waste heat recovery technology, and mash-to-mash heat exchange technology are used, with cooperation of high-alcohol-content fermentation, the energy consumption is significantly reduced to a highest extent where the overall steam consumption may be lower than 3.0 tons/ton of alcohol, which is 15-30% lower than the current ethanol fuel using the starch as a raw material.
4, The present invention has no special requirements on Elaeagnus angustifolia L. as the raw materials, may effectively broaden the use of Elaeagnus angustifolia L., and make full use of the Elaeagnus angustifolia L. resources.
5, In the process of the present invention, the liquefaction treatment section and the solid-liquid treatment are processes of separating insoluble matter such as jujube skin and fiber from the jujube pulp. As the jujube skin and fiber in the jujube pulp still contain 5% to 10% sugar, the solid-liquid separation adopts multiple grinding, washing, and separation to soften the jujube skin and fiber in the jujube pulp to reduce the residual sugar in the insoluble solids, so that the sugar is fully released, and the problems of blocking the distillation section caused by wearing the Elaeagnus angustifolia L. skin and fiber in the distillation section is also avoided.
6, In the process of the present invention, the liquefaction treatment section adopts the addition of pectinase and other viscosity reducing enzymes, and then performs cooking treatment, solid-liquid separation, multi-effect evaporation and concentration. Since the jujube skin and fiber in the jujube pulp still contain 5-10% of sugar, the method of cooking may soften the skin and fiber, dissolve the solid sugar state into a liquid sugar state, so as to fully release the sugar. Adding pectinase may reduce the viscosity of jujube pulp and reduce the residue of sugar in insoluble solids, so that the sugar is fully released, and the problem of sharply risen energy consumption of the fermentation and distillation sections caused by poor viscosity and fluidity of the jujube pulp is also avoided. The use of the liquefaction treatment may increase the sugar yield in the Elaeagnus angustifolia L. by 5-9%, greatly improve the utilization rate of raw materials, and reduce the cost of raw materials consumption.
7, For the method of the present invention, the sugar-alcohol conversion rate and production efficiency is basically the same as when fermented with corn and other starches as raw materials, so that a new biological resource to produce bioenergy is used without consuming food resources, which broadens the source of raw materials for bioenergy.

### BRIEF DESCRIPTION OF DRAWINGS

FIG. 1 is a flow diagram of Embodiment 1 of a process for producing ethanol fuel by using Elaeagnus angustifolia L. as a raw material;

FIG. 2 is a flow diagram of Embodiment 2 of a process for producing ethanol fuel by using Elaeagnus angustifolia L. as a raw material;

FIG. 3 is a flow chart of Embodiment 1 of a process for producing ethanol fuel by using Elaeagnus angustifolia L. as a raw material;

FIG. 4 is a flow chart of Embodiment 2 of a process for producing ethanol fuel by using Elaeagnus angustifolia L. as a raw material;

FIG. 5 is a flow chart of Embodiment 3 of a process for producing ethanol fuel by using Elaeagnus angustifolia L. as a raw material;

FIG. 6 is a flow chart of Embodiment 4 of a process for producing ethanol fuel by using Elaeagnus angustifolia L. as a raw material;

FIG. 7 is a simplified flow chart of Embodiment 5 of a process for producing ethanol fuel by using Elaeagnus angustifolia L. as a raw material;

FIG. 8 is a flow chart of Embodiment 5 of a process for producing ethanol fuel by using Elaeagnus angustifolia L. as a raw material.

### DETAIL DESCRIPTIONS OF EMBODIMENTS

### Comparative Example 1

Comparative Example 1 is a process for fermenting and producing ethanol fuel by using corn as a raw material:

143.2 kg of corn (with a starch content of 65% and a moisture content of 14%) is crushed into a pre-liquefaction tank, mixing water and α-amylase are added, to obtain a slurry with a dry matter content of 26.5%, wherein in the pre-liquefaction tank, the above slurry is heated to 80° C. to 90° C. by direct steam;

then the slurry is pumped to a jet liquefier, and a discharge temperature of a steam ejector is controlled to be 95° C. to 105° C. to obtain a heated mash; the heated mash is sent to a cooking column for cooking and ripening; the ripened mash is cooled down to 60° C. to 65° C. to obtain a liquefied mash;

the liquefied mash is adjusted with sulfuric acid in a saccharification tank (equipped with agitator) to adjust the pH value, and then saccharification enzyme is added for saccharification; the saccharified mash is conveyed by a pump and cooled step by step through a two-stage plate cooler, cooled to 28° C. to 33° C., and sent to a fermentation section;

the fermentation section adopts batch fermentation, and the mash is added with the yeast from a barmy tank, wherein the fermentation time is 60 h, and the temperature is 33° C.; after fermentation, the fermented and matured mash is sent to a distillation and dehydration section;

an alcohol vapor (95.8% V/V) obtained after the fermentation of the matured mash is distilled; the waste mash is sent to a waste processing section; the alcohol vapor is sent to a molecular sieve dehydration section to obtain a product vapor of anhydrous ethanol, and 43.0 kg of ethanol fuel product is obtained after the product vapor of anhydrous ethanol is condensed; the product mass fraction is 99.5%, and the water content is <0.5%, which meets the national standard for denatured ethanol fuel GB18350-2013;

the waste mash obtained after distillation is treated by solid-liquid separation; part of the separated clear liquid is reused as water for the liquid chemical section, and the rest is fed to the evaporation concentration unit to produce a thick slurry; the thick slurry is mixed with solid residue, dried, granulated, and cooled to obtain DDGS fodder with a moisture content of less than 11.5%. The total steam consumption of the whole section is 3.9 to 4.0 tons/ton of the ethanol fuel (including the comprehensive treatment section of waste grains).

### Embodiment 1

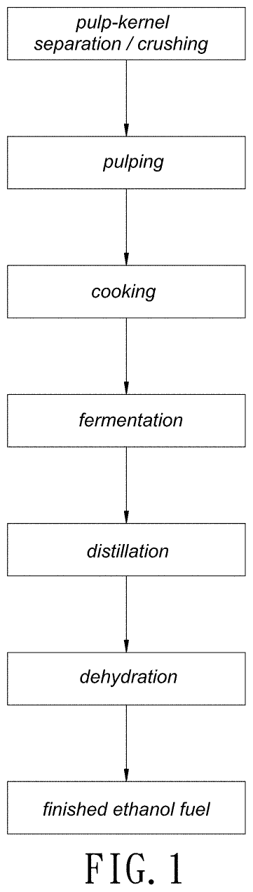

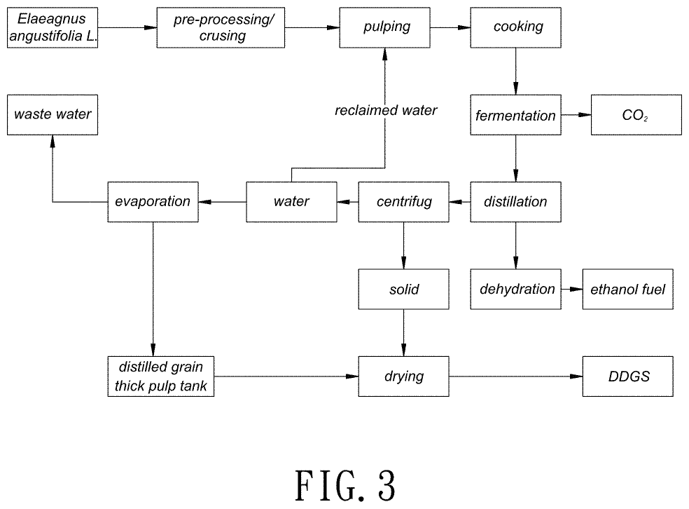

With reference to FIGS. 1 and 3, for Embodiment 1, a process for producing ethanol fuel by using Elaeagnus angustifolia L. as a raw material is as follows:

40 Kg of Elaeagnus angustifolia L. powder with removed impurities and kernel is adding with process water with a pH of 3.5 to 5.0 for pulping to obtain a jujube pulp, and a mass concentration is controlled to and a sugar degree of the jujube pulp is controlled to 20% to 30% and to about 15% to 17%;

the jujube pulp is heated to 85° C. to 100° C. through a multi-stage ejector and sent to a cooking kettle for cooking and keeping warm for 50 min to 90 min; the jujube pulp mash is cooled down to 28° C. to 35° C. through a heat exchanger;

the jujube pulp clear mash is added directly into the fermentation tank, and the yeast is inoculated for stirring and fermenting for 36 h to 40h under 30° C. to 34° C.; alcohol is generated through the yeast in the fermentation tank, and a sterile air is passed through the fermentation process to maintain the vitality of the yeast, and the fermentation is stopped when the residual sugar concentration of the mash is lower than 0.5%, so as to obtain a matured mash;

the matured mash is preheated to above 70° C. and sending to the distillation system, and then an alcohol is extracted with a volume fraction of 95% from a liquid phase of a rectification tower as an anhydrous raw material after differential distillation;

the anhydrous raw material is preheated and sent to a molecular sieve system for dehydration after passing through an evaporator and a superheater to obtain an ethanol fuel gas, and then cooling the ethanol fuel gas to obtain 9.04 kg of an ethanol fuel with a mass fraction of 99.5%; in the cooling process of ethanol fuel gas, the waste heat generated during the cooling process of ethanol fuel gas is recovered, and the recovered waste heat may be used to preheat anhydrous raw materials to realize heat recovery and utilization and reduce costs; the steam consumption in the distillation and dehydration section is 1.4-1.6 tons/ton of ethanol fuel;

a deanaturing agent is added into the ethanol fuel with a mass fraction of 99.5% to obtain the finished ethanol fuel, wherein the product quality meets the national standard GB18350-2013 (shown in Table 1);

TABLE 1
Test results for ethanol fuel

| | | | GB 18350-2013 | Test |
|---|---|---|---|---|
| No. | Item | Unit | index | results |
| 1 | appearance | | clear and | qualified |
| | | | transparent, without | |
| | | | visible suspended | |
| | | | matter and | |
| | | | sediment | |
| 2 | ethanol | % | ≥92.1 | 99.2 |
| 3 | moisture | % | ≤0.8 | 0.51 |
| 4 | methanol | % | ≤0.5 | 0.08 |
| 5 | solvent washed | mg/100 ml | ≤5.0 | 4.2 |
| | gum | | | |
| 6 | inorganic chlorine | mg/L | ≤8 | 3 |
| | (calculated by cl−) | | | |
| 7 | acidity (calculated | mg/L | ≤56 | 30.95 |
| | as acetic acid) | | | |
| 8 | Copper | mg/L | ≤0.08 | 0.05 |
| 9 | PHe | mg/L | 6.5-9.0 | 7.33 |
| 10 | sulfur | mg/kg | 30^{a}/10^{b} | <10 |

The waste mash discharged from the distillation dehydration system undergoes the solid-liquid separation, evaporation and concentration, and part of the wastewater is reused as reclaimed water. DDG is produced by drying solid distillers grains or mixed with liquid distillers grains after evaporation to produce DDGS. The total steam consumption of the whole section is 3.4 to 3.6 tons/ton of the ethanol fuel (including the comprehensive treatment section of waste grains).

Compared with the process of Comparative Example 1, the process is similar to the process of producing ethanol fuel by using corn and other starch as raw materials. The process technology is reliable without adding amylase and glucoamylase during the cooking and sterilization process, which saves production costs.

### Embodiment 2

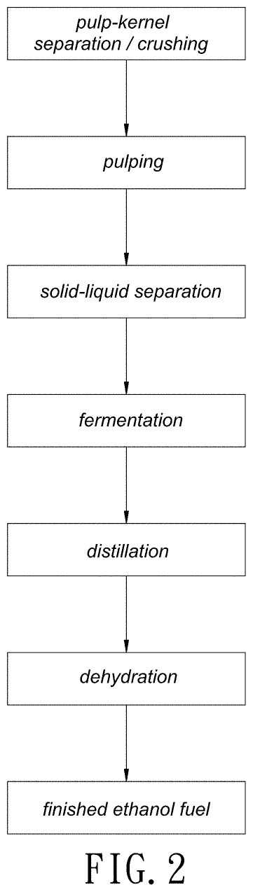

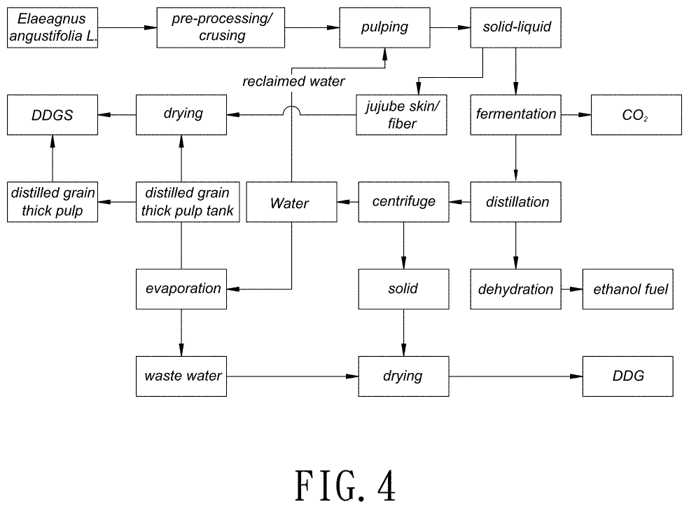

With reference to FIGS. 2 and 4, for Embodiment 2, a process for producing ethanol fuel by using Elaeagnus angustifolia L. as a raw material is as follows:

30 Kg of Elaeagnus angustifolia L. powder with removed impurities and kernel is adding with process water with a pH of 3.5 to 5.0 for pulping to obtain a jujube pulp, and a mass concentration is controlled to and a sugar degree of the jujube pulp is controlled to 20% to 30% and to about 10% to 15%;

after solid-liquid separation of the jujube pulp, directly sending a pure mash to a fermentation tank; after separation, composting or drying the solid to produce a fodder;

the clear mash is added directly into the fermentation tank, and the yeast is inoculated and an antibacterial agent is added for stirring and fermenting for 36 h to 40h under 30° C. to 34° C.; alcohol is generated through the yeast in the fermentation tank, and a sterile air is passed through the fermentation process to maintain the vitality of the yeast, and the fermentation is stopped when the residual sugar concentration of the mash is lower than 0.5%, so as to obtain a matured mash;

the matured mash is preheated to above 70° C. and sent to the distillation workshop, and then an alcohol is extracted with a volume fraction of 95% from a liquid phase of a rectification tower as an anhydrous raw material after differential distillation;

the anhydrous raw material is preheated and sent to a molecular sieve system for dehydration after passing through an evaporator and a superheater to obtain an ethanol fuel gas, and then cooling the ethanol fuel gas to obtain 7.85 kg of an ethanol fuel with a mass fraction of 99.5%; in the cooling process of ethanol fuel gas, the waste heat generated during the cooling process of ethanol fuel gas is recovered, and the recovered waste heat may be used to preheat anhydrous raw materials to realize heat recovery and utilization and reduce costs;

a deanaturing agent is added into the ethanol fuel with a mass fraction of 99.5% to obtain the finished ethanol fuel, wherein the product quality meets the national standard GB18350-2013 (shown in Table 2);

TABLE 2
Test results for ethanol fuel

| | | | GB 18350-2013 | Test |
|---|---|---|---|---|
| No. | Item | Unit | index | results |
| 1 | appearance | | clear and | qualified |
| | | | transparent, without | |
| | | | visible suspended | |
| | | | matter and | |
| | | | sediment | |
| 2 | ethanol | % | ≥92.1 | 99.3 |
| 3 | moisture | % | ≤0.8 | 0.50 |
| 4 | methanol | % | ≤0.5 | 0.09 |
| 5 | solvent washed | mg/100 ml | ≤5.0 | 4.0 |
| | gum | | | |
| 6 | inorganic chlorine | mg/L | ≤8 | 2 |
| | (calculated by cl−) | | | |
| 7 | acidity (calculated | mg/L | ≤56 | 33.95 |
| | as acetic acid) | | | |
| 8 | Copper | mg/L | ≤0.08 | 0.04 |
| 9 | PHe | mg/L | 6.5-9.0 | 7.33 |
| 10 | sulfur | mg/kg | 30^{a}/10^{b} | <10 |

The waste mash discharged from the distillation dehydration system undergoes the solid-liquid separation, evaporation and concentration, and part of the wastewater is reused as reclaimed water. DDG is produced by drying solid distillers grains or mixed with liquid distillers grains after evaporation to produce DDGS.

Compared with starch raw materials such as corn in Comparative Example 1, Elaeagnus angustifolia L. raw material contains higher directly fermentable sugars, so that the raw material fermentation method is adopted to carry out solid-liquid separation of the Elaeagnus angustifolia L. pulp, and then fermented according to the clear liquid fermentation process.

The process advantage of the present embodiment is that the mash does not need to be cooked, and the clear liquor fermentation process is used, so that the process is simple, and with the waste heat recovery technology, the steam consumption of ethanol fuel is less than 3.0 tons/ton alcohol while the overall steam consumption reaching or even lowering the steam consumption level of traditional process of producing ethanol fuel by using corn, thereby leading the cost of the production process to be lower than that of the process of producing ethanol fuel by using corn and cassava as raw materials.

### Embodiment 3

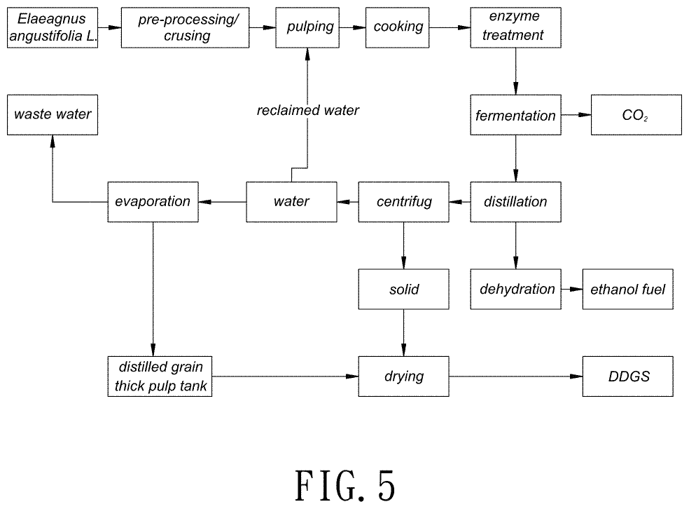

With reference to FIG. 5, for Embodiment 3, a process for producing ethanol fuel by using Elaeagnus angustifolia L. as a raw material is as follows:

220 Kg of Elaeagnus angustifolia L. powder with removed impurities and kernel is adding with process water with nutrients with a pH of 3.5 to 5.0 for pulping to obtain a jujube pulp, and a mass concentration is controlled to and a sugar degree of the jujube pulp is controlled to 20% to 35% and to about 16% to 20%;

the jujube pulp is preheated to 30° C. to 60° C. through a first-stage ejector, adding pectinase in a proportion of 0.05% to 0.1% and keeping warm for 1 hour to 2 hours, then heating the jujube pulp to 85° C. to 100° C. through a multi-stage ejector and sending to a cooking kettle for cooking and keeping warm for 30 min to 180 min; the jujube pulp mash and the unpreheated jujube mash are exchanged mash-to-mash for heat, the jujube mash is preheated, and then the jujube pulp mash is cooled to 20° C. to 35° C. by a cooling water heat exchanger;

the jujube pulp clear mash is added directly into the fermentation tank, and the yeast is inoculated for fermenting for 36 h to 40 h; alcohol is generated through the yeast in the fermentation tank, and a sterile air is passed through the fermentation process to maintain the vitality of the yeast, wherein the final alcohol content of the fermentation is 11-14% (V/V) and the residual sugar concentration is less than 0.5%, so as to obtain a matured mash;

the matured mash is preheated to above 70° C. and sent to the distillation workshop, and then an alcohol is extracted with a volume fraction of 95% from a liquid phase of a rectification tower as an anhydrous raw material after differential distillation;

the anhydrous raw material is preheated and sent to a molecular sieve system for dehydration after passing through an evaporator and a superheater to obtain an ethanol fuel gas, and then cooling the ethanol fuel gas to obtain 54 kg of an ethanol fuel with a mass fraction of 99.5%; in the cooling process of ethanol fuel gas, the waste heat generated during the cooling process of ethanol fuel gas is recovered, and the recovered waste heat may be used to preheat subsequent anhydrous raw materials to realize heat recovery and utilization and reduce costs;

a deanaturing agent is added into the ethanol fuel with a mass fraction of 99.5% to obtain the finished ethanol fuel, wherein the product quality meets the national standard GB18350-2013 (shown in Table 3);

TABLE 3
Test results for ethanol fuel

| | | | GB 18350-2013 | Test |
|---|---|---|---|---|
| No. | Item | Unit | index | results |
| 1 | appearance | | clear and | qualified |
| | | | transparent, without | |
| | | | visible suspended | |
| | | | matter and | |
| | | | sediment | |
| 2 | ethanol | % | ≥92.1 | 99.5 |
| 3 | moisture | % | ≤0.8 | 0.60 |
| 4 | methanol | % | ≤0.5 | 0.10 |
| 5 | solvent washed | mg/100 ml | ≤5.0 | 4.1 |
| | gum | | | |
| 6 | inorganic chlorine | mg/L | ≤8 | 5 |
| | (calculated by cl−) | | | |
| 7 | acidity (calculated | mg/L | ≤56 | 33.90 |
| | as acetic acid) | | | |
| 8 | Copper | mg/L | ≤0.08 | 0.04 |
| 9 | PHe | mg/L | 6.5-9.0 | 7.53 |
| 10 | sulfur | mg/kg | 30^{a}/10^{b} | <10 |

The waste mash discharged from the distillation dehydration system undergoes the solid-liquid separation, evaporation and concentration, and part of the wastewater is reused as reclaimed water. DDG is produced by drying solid distillers grains or mixed with liquid distillers grains after evaporation to produce DDGS.

The process advantages of the present embodiment are: the jujube pulp undergoes fermentation as a whole after enzymatic hydrolysis, which may well solve the fluidity problem of the mash; the pipeline may not be blocked, and the sugar in the raw material is completely released; the sugar alcohol conversion rate is high, and the sugar alcohol conversion rate is greater than 45%. After cooking and enzymatic treatment, under the condition that the sugar content of the fermented mash is maintained at more than 16%, the sugar yield in the Elaeagnus angustifolia L. may be increased by 2.5% to 4.0% compared with Embodiment 1.

### Embodiment 4

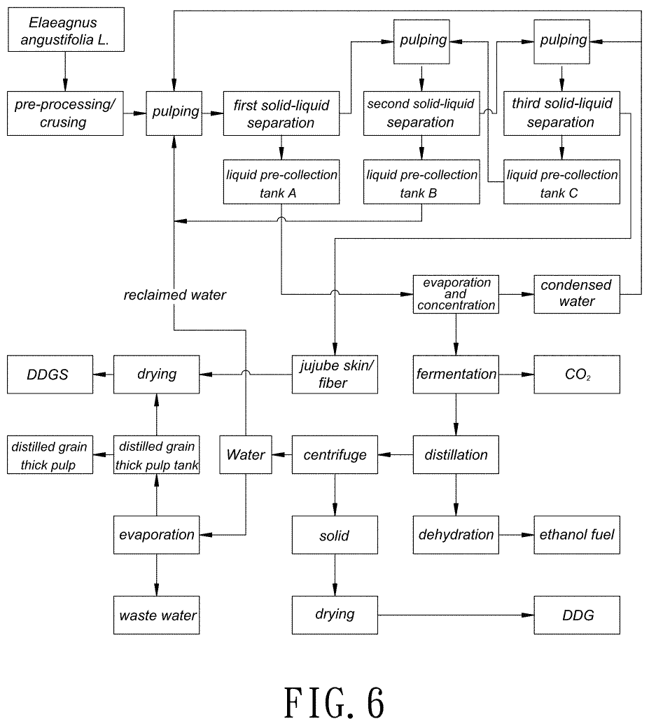

With reference to FIG. 6, for Embodiment 4, a process for producing ethanol fuel by using Elaeagnus angustifolia L. as a raw material is as follows:

175 Kg of Elaeagnus angustifolia L. powder with removed impurities and kernel is adding with process water with a pH of 3.5 to 5.0 for pulping to obtain a jujube pulp, and a mass concentration is controlled to and a sugar degree of the jujube pulp is controlled to 15% to 30% and to about 15%;

after the jujube is subjected to the solid-liquid separation treatment such as triple pulping, washing, separation (as shown in FIG. 6), the clear liquid is collected and concentrated by multi-effect evaporation, wherein before the clear liquid enters the evaporator, it is preheated by the waste heat recovered in the distillation dehydration section, and then enters the multi-effect evaporator for evaporation and concentration; the concentrated mash is directly sent to the fermentation tank; the separated solid is composted or dried to produce fodder;

the clear mash is added directly into the fermentation tank, and the yeast is inoculated and an antibacterial agent is added for fermenting for 36 h to 40 h; alcohol is generated through the yeast in the fermentation tank, and a sterile air is passed through the fermentation process to maintain the vitality of the yeast, wherein the final alcohol content of the fermentation is 8-9% (V/V) and the residual sugar concentration is less than 0.5%, so as to obtain a matured mash;

the matured mash is preheated to above 70° C. and sent to the distillation workshop, and then an alcohol is extracted with a volume fraction of 95% from a liquid phase of a rectification tower as an anhydrous raw material after differential distillation;

the anhydrous raw material is preheated and sent to a molecular sieve system for dehydration after passing through an evaporator and a superheater to obtain an ethanol fuel gas, and then cooling the ethanol fuel gas to obtain 41 kg of an ethanol fuel with a mass fraction of 99.5%; in the cooling process of ethanol fuel gas, the waste heat generated during the cooling process of ethanol fuel gas is recovered, and the recovered waste heat may be used to preheat anhydrous raw materials to realize heat recovery and utilization and reduce costs;

a deanaturing agent is added into the ethanol fuel with a mass fraction of 99.5% to obtain the finished ethanol fuel, wherein the product quality meets the national standard GB18350-2013 (shown in Table 4);

TABLE 4
Test results for ethanol fuel

| | | | GB 18350-2013 | Test |
|---|---|---|---|---|
| No. | Item | Unit | index | results |
| 1 | appearance | | clear and | qualified |
| | | | transparent, without | |
| | | | visible suspended | |
| | | | matter and | |
| | | | sediment | |
| 2 | ethanol | % | ≥92.1 | 99.4 |
| 3 | moisture | % | ≤0.8 | 0.50 |
| 4 | methanol | % | ≤0.5 | 0.06 |
| 5 | solvent washed | mg/100 ml | ≤5.0 | 4.2 |
| | gum | | | |
| 6 | inorganic chlorine | mg/L | ≤8 | 4 |
| | (calculated by cl−) | | | |
| 7 | acidity (calculated | mg/L | ≤56 | 35.90 |
| | as acetic acid) | | | |
| 8 | Copper | mg/L | ≤0.08 | 0.06 |
| 9 | PHe | mg/L | 6.5-9.0 | 7.30 |
| 10 | sulfur | mg/kg | 30^{a}/10^{b} | <10 |

The waste mash discharged from the distillation dehydration system undergoes the solid-liquid separation, evaporation and concentration, and part of the wastewater is reused as reclaimed water. DDG is produced by drying solid distillers grains or mixed with liquid distillers grains after evaporation to produce DDGS.

After the solid-liquid separation, grinding, washing and multi-effect evaporation, in the case of the sugar content of the fermented mash maintaining above 15%, the sugar yield in the Elaeagnus angustifolia L. may be increased by 2.0 to 5.0% compared to Embodiment 1; the mash is directly fermented with raw materials without cooking, the waste heat is recovered from the waste steam in the distillation and dehydration section, and after the solid-liquid separation for the jujube pulp, the clear liquid is heated, evaporated and concentrated while preheating the matured mash, so that the steam consumption may be reduced by 20% to 30%, and the steam consumption in the whole process is 2.8 to 3.0 tons/ton of ethanol fuel. Since the process adopts the raw material fermentation process, the process does not need to add enzyme preparations, so that the production cost is greatly saved.

### Embodiment 5

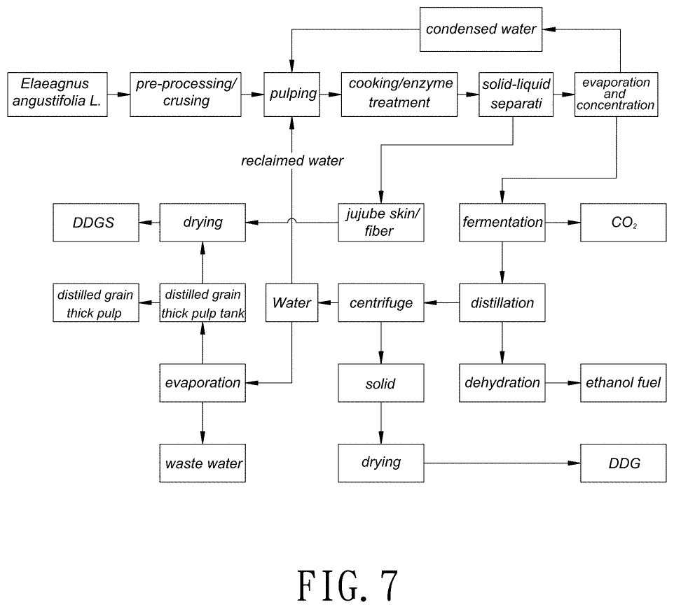

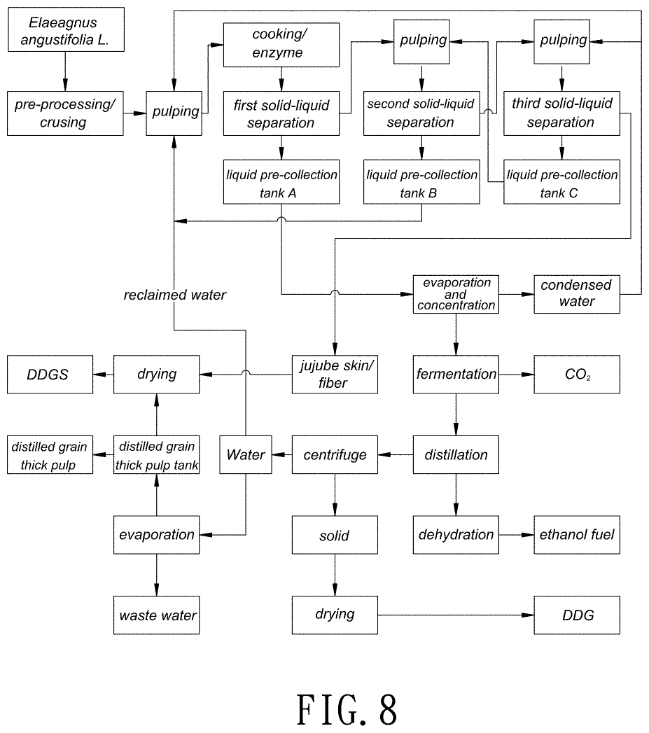

With reference to FIGS. 7 and 8, for Embodiment 5, a process for producing ethanol fuel by using Elaeagnus angustifolia L. as a raw material is as follows:

220 Kg of Elaeagnus angustifolia L. powder with removed impurities and kernel is adding with process water with nutrients with a pH of 3.5 to 5.0 for pulping to obtain a jujube pulp, and a mass concentration is controlled to 20% to 35%;

the jujube pulp is preheated to 30° C. to 60° C. by the heat exchanger through the steam waste heat from the distillation and dehydration section, pectinase is added in a proportion of 0.05% to 0.1% while keeping warm for 1 hour to 2 hours, then the jujube pulp is heated to 85° C. to 110° C. through a multi-stage ejector and sent to a cooking kettle for cooking and keeping warm for 30 min to 180 min;

after many times of grinding, washing and separation of the jujube pulp, the clear liquid is collected, and is subjected to multi-effect evaporation and concentration, to concentrate the clear liquid to have a sugar content of 16% to 24%; the concentrated clear mash is directly sent to the fermentation tank; after separation, the solid is composted and dried to produce a fodder;

the clear mash is subjected to mash-to-mash heat exchange with the matured mash through then heat exchanger, and then cooled down to 20° C. to 35° C. through heat exchange with cooling water; the clear mash is sent directly into the fermentation tank, and the yeast is inoculated for fermenting for 36 h to 48 h; alcohol is generated through the yeast in the fermentation tank, and a sterile air is passed through the fermentation process to maintain the vitality of the yeast, wherein the final alcohol content of the fermentation is 11-14% (V/V) and the residual sugar concentration is less than 0.5%, so as to obtain a matured mash;

the matured mash is preheated to above 70° C. and sent to the distillation workshop through mash-to-mash heat exchange, and then an alcohol is extracted with a volume fraction of 95% from a liquid phase of a rectification tower as an anhydrous raw material after differential distillation;

the anhydrous raw material is preheated and sent to a molecular sieve system for dehydration after passing through an evaporator and a superheater to obtain an ethanol fuel gas, and then cooling the ethanol fuel gas to obtain 57 kg of an ethanol fuel with a mass fraction of 99.5%; in the cooling process of ethanol fuel gas, the waste heat generated during the cooling process of ethanol fuel gas is recovered, and the recovered waste heat may be used to preheat anhydrous raw materials to realize heat recovery and utilization and reduce costs;

a deanaturing agent is added into the ethanol fuel with a mass fraction of 99.5% to obtain the finished ethanol fuel, wherein the product quality meets the national standard GB18350-2013 (shown in Table 5);

TABLE 5
Test results for ethanol fuel

| | | | GB 18350-2013 | Test |
|---|---|---|---|---|
| No. | Item | Unit | index | results |
| 1 | appearance | | clear and | qualified |
| | | | transparent, without | |
| | | | visible suspended | |
| | | | matter and | |
| | | | sediment | |
| 2 | ethanol | % | ≥92.1 | 99.4 |
| 3 | moisture | % | ≤0.8 | 0.50 |
| 4 | methanol | % | ≤0.5 | 0.08 |
| 5 | solvent washed | mg/100 ml | ≤5.0 | 4.1 |
| | gum | | | |
| 6 | inorganic chlorine | mg/L | ≤8 | 5 |
| | (calculated by cl−) | | | |
| 7 | acidity (calculated | mg/L | ≤56 | 33.92 |
| | as acetic acid) | | | |
| 8 | Copper | mg/L | ≤0.08 | 0.07 |
| 9 | PHe | mg/L | 6.5-9.0 | 7.52 |
| 10 | sulfur | mg/kg | 30^{a}/10^{b} | <10 |

The waste mash discharged from the distillation dehydration system undergoes the solid-liquid separation, evaporation and concentration, and part of the wastewater is reused as reclaimed water. DDG is produced by drying solid distillers grains or mixed with liquid distillers grains after evaporation to produce DDGS.

After cooking, the solid-liquid separation, grinding, washing and multi-effect evaporation, under the condition that the sugar content of the fermented mash is maintained at more than 16%, the sugar yield in the Elaeagnus angustifolia L. may be increased by 5.0% to 9.0% compared with Embodiment 1.

The above embodiments are preferred embodiments of the present invention, but the embodiments of the present invention are not limited by the above embodiments. Any other changes, modifications, substitutions, combinations, and simplifications that do not depart from the spirit and principle of the present invention should be equivalent replacement methods and are all included in the protection scope of the present invention.

1. A process method for producing ethanol fuel by using Elaeagnus angustifolia L. as a raw material, comprising steps of pretreatment and pulping of Elaeagnus angustifolia L., liquefaction treatment, fermentation, distillation and dehydration of ethanol, wherein
(1) pretreatment and pulping, including enucleating Elaeagnus angustifolia L., adding process water with a pH of 3.5 to 5.0 for pulping to obtain a jujube pulp, and controlling a mass concentration of the jujube pulp to 15% to 30%;
(2) liquefaction treatment, including performing a liquefaction treatment on the jujube pulp;
(3) fermentation, including inoculating an active dry yeast, fermenting to obtain a fermented and matured mash;
(4) distillation and dehydration of ethanol, including distilling and dehydrating the matured mash, then condensing to obtain an ethanol liquid, followed by adding a deanaturing agent, to obtain a finished ethanol fuel;
the distillation and dehydration of ethanol specifically includes:
preheating the matured mash to above 70° C. and sending to the distillation workshop, and then extracting an alcohol with a volume fraction of 95% from a liquid phase of a rectification tower as an anhydrous raw material after differential distillation;
preheating the anhydrous raw material and sending to a molecular sieve system for dehydration after passing through an evaporator and a superheater to obtain an ethanol fuel gas, and then cooling the ethanol fuel gas to obtain an ethanol fuel with a mass fraction of 99.5%;
adding a deanaturing agent into the ethanol fuel with a mass fraction of 99.5% to obtain the finished ethanol fuel;
the preheating the matured mash may include primary steam preheating directly or preheating with waste heat recovered from the distillation and dehydration section.

2. The method for producing ethanol fuel by using Elaeagnus angustifolia L. as a raw material according to claim 1, wherein the liquefaction treatment comprises steps of:
after solid-liquid separation of the jujube pulp, directly sending a pure mash to a fermentation tank; after separation, composting or drying the solid to produce a fodder.

3. The method for producing ethanol fuel by using Elaeagnus angustifolia L. as a raw material according to claim 1, wherein the liquefaction treatment comprises steps of:
preheating the jujube pulp to 30° C. to 60° C. through a first-stage ejector, adding pectinase in a proportion of 0.05% to 0.1% and keeping warm for 1 hour to 2 hours, then heating the jujube pulp to 85° C. to 105° C. through a multi-stage ejector and sending to a cooking kettle for cooking and keeping warm for 30 min to 180 min, to obtain the jujube pulp mash; cooling the jujube pulp mash to 20° C. to 35° C. through a heat exchanger.

4. The method for producing ethanol fuel by using Elaeagnus angustifolia L. as a raw material according to claim 1, wherein the liquefaction treatment employs a continuous cooking process of cooking after multi-stage low-pressure steam ejection, so that the mixed pulp is evenly heated; the multi-stage steam ejection is first-stage or more-stage steam ejection.

5. The method for producing ethanol fuel by using Elaeagnus angustifolia L. as a raw material according to claim 2, wherein a time of the mash in the fermentation in the fermentation tank is 36 hours to 60 hours; an alcohol content of the fermented and matured mash reaches 7-11% (v/v); during the fermentation process, heat will be released, and a fermentation temperature may be stabilized by cooling outside the tank; the fermentation temperature is 28° C. to 35° C.; the fermentation tank employs a vertical agitator.

6. The method for producing ethanol fuel by using Elaeagnus angustifolia L. as a raw material according to claim 2, further comprising a comprehensive waste treatment section;
a treatment process of the comprehensive waste treatment section comprises performing solid-liquid separation of wastes discharged from the distillation and dehydration sections of ethanol, wherein the separated wet wastes are used as solid biological fertilizers or dried, and the separated clear liquid is partly reused and partly enters a liquid fertilizer storage tank.

7. The method for producing ethanol fuel by using Elaeagnus angustifolia L. as a raw material according to claim 1, further comprising subsequent byproduct recovery and reuse sections, wherein the subsequent byproduct recovery and reuse sections comprise recovery of liquid carbon dioxide, production, refinement and purification of biogas, and precision separation of fuel oil.

8. A process method for producing ethanol fuel by using Elaeagnus angustifolia L. as a raw material, comprising steps of pretreatment and pulping of Elaeagnus angustifolia L., liquefaction treatment, fermentation, distillation and dehydration of ethanol, wherein
(1) pretreatment and pulping, including enucleating Elaeagnus angustifolia L., adding process water with a pH of 3.5 to 5.0 for pulping to obtain a jujube pulp, and controlling a mass concentration of the jujube pulp to 15% to 30%;
(2) liquefaction treatment, including performing a liquefaction treatment on the jujube pulp;
(3) fermentation, including inoculating an active dry yeast, fermenting to obtain a fermented and matured mash;
(4) distillation and dehydration of ethanol, including distilling and dehydrating the matured mash, then condensing to obtain an ethanol liquid, followed by adding a deanaturing agent, to obtain a finished ethanol fuel.

9. The method for producing ethanol fuel by using Elaeagnus angustifolia L. as a raw material according to claim 8, wherein the liquefaction treatment comprises steps of:
after solid-liquid separation of the jujube pulp, directly sending a pure mash to a fermentation tank; after separation, composting or drying the solid to produce a fodder;
wherein the solid-liquid separation is a process of beating, grinding, washing, and separating one or more times.

10. The method for producing ethanol fuel by using Elaeagnus angustifolia L. as a raw material according to claim 8, wherein the solid-liquid separation comprises multiple grinding, washing, and separating using a filter press and/or a butterfly centrifuge and/or a double cone centrifuge and/or a decanter centrifuge.

11. The method for producing ethanol fuel by using Elaeagnus angustifolia L. as a raw material according to claim 8, wherein the liquefaction treatment comprises: preheating the jujube pulp to 30° C. to 60° C. through a first-stage ejector, adding pectinase in a proportion of 0.05% to 0.1% and keeping warm for 1 hour to 2 hours, then heating the jujube pulp to 85° C. to 105° C. through a multi-stage ejector and sending to a cooking kettle for cooking and keeping warm for 30 min to 180 min; performing multiple grinding, washing, and separating on the jujube pulp, collecting the clear liquid, concentrating by multi-effect evaporation, and sending the concentrated mash directly to the fermentation tank, wherein after the separation, the solids are composted or dried to produce a fodder;
the preheating the jujube pulp may employ one of:
primary steam preheating;
heat exchange of the mash with waste heat recovered from the cooling process of the mash;
preheating waste heat recovered from the distillation and dehydration section.

12. The method for producing ethanol fuel by using Elaeagnus angustifolia L. according to claim 8, wherein the distillation and dehydration of ethanol specifically comprises:
preheating the matured mash to above 70° C. and sending to the distillation workshop, and then extracting an alcohol with a volume fraction of 95% from a liquid phase of a rectification tower as an anhydrous raw material after differential distillation;
preheating the anhydrous raw material and sending to a molecular sieve system for dehydration after passing through an evaporator and a superheater to obtain an ethanol fuel gas, and then cooling the ethanol fuel gas to obtain an ethanol fuel with a mass fraction of 99.5%;
adding a deanaturing agent into the ethanol fuel with a mass fraction of 99.5% to obtain the finished ethanol fuel;
the preheating the matured mash may include primary steam preheating directly or preheating with waste heat recovered from the distillation and dehydration section.

13. The method for producing ethanol fuel by using Elaeagnus angustifolia L. as a raw material according to claim 9, wherein a time of the mash in the fermentation in the fermentation tank is 36 hours to 60 hours; an alcohol content of the fermented and matured mash reaches 9-13% (v/v); during the fermentation process, heat will be released, and a fermentation temperature may be stabilized by cooling outside the tank; the fermentation temperature is 28° C. to 35° C.; the fermentation tank employs an agitator.

14. The method for producing ethanol fuel by using Elaeagnus angustifolia L. as a raw material according to claim 9 or 10, further comprising a comprehensive waste treatment section;
a treatment process of the comprehensive waste treatment section comprises performing solid-liquid separation of wastes discharged from the distillation and dehydration sections of ethanol, wherein the separated wet wastes are used as solid biological fertilizers or dried, and the separated clear liquid is partly reused and partly enters a liquid fertilizer storage tank.

15. The method for producing ethanol fuel by using Elaeagnus angustifolia L. as a raw material according to claim 8, further comprising subsequent byproduct recovery and reuse sections;
the subsequent byproduct recovery and reuse sections comprise recovery of liquid carbon dioxide, production, refinement and purification of biogas, and precision separation of fuel oil.

16. The method for producing ethanol fuel by using Elaeagnus angustifolia L. as a raw material according to claim 10, wherein a time of the mash in the fermentation in the fermentation tank is 36 hours to 60 hours; an alcohol content of the fermented and matured mash reaches 9-13% (v/v); during the fermentation process, heat will be released, and a fermentation temperature may be stabilized by cooling outside the tank; the fermentation temperature is 28° C. to 35° C.; the fermentation tank employs an agitator.

17. The method for producing ethanol fuel by using Elaeagnus angustifolia L. as a raw material according to claim 10, further comprising a comprehensive waste treatment section;
a treatment process of the comprehensive waste treatment section comprises performing solid-liquid separation of wastes discharged from the distillation and dehydration sections of ethanol, wherein the separated wet wastes are used as solid biological fertilizers or dried, and the separated clear liquid is partly reused and partly enters a liquid fertilizer storage tank.
